(19) United States
(12) Patent Application Publication
(10) Pub. No.: US 2018/0113991 A1
(43) Pub. Date: Apr. 26, 2018

(54) Interactive Apparatus and Devices for Personal Symptom Management and Therapeutic Treatment Systems

(71) Applicant: Sarah Davidson, Ponte Vedra Beach, FL (US)

(72) Inventor: Sarah Davidson, Ponte Vedra Beach, FL (US)

(21) Appl. No.: 15/793,931

(22) Filed: Oct. 25, 2017

Related U.S. Application Data
(60) Provisional application No. 62/413,427, filed on Oct. 26, 2016.

(51) Int. Cl. G06F 19/00; A61B 5/00

(52) U.S. Cl. CPC G06F 19/3431; G06F 19/3406; A61B 5/0022; G06F 19/3418; G06F 19/3481

(57) ABSTRACT

The present disclosure relates generally to symptom management. More specifically, the disclosure describes a system that pairs biomonitoring mechanisms with a symptom management system. In some aspects, the symptom management system may comprise customized programming that may provide engaging user activities that may directly or indirectly affect one or more symptoms. In some embodiments, a user may be diagnosed with a disease, infection, or disorder, and the symptom management system may manage and monitor a predefined set of symptoms associated with the diagnosis. In some implementations, individual symptoms may be identified and subsequently monitored and managed separately, such as through the creation and subsequent tracking of a symptom management score.

### CROSS-REFERENCE TO RELATED APPLICATION

This application claims priority to and the full benefit of U.S. Provisional Patent Application Ser. No. 62/413,427, filed Oct. 26, 2016, and titled “INTERACTIVE APPARATUS AND DEVICES FOR PERSONAL SYMPTOM MANAGEMENT AND THERAPEUTIC TREATMENT SYSTEMS”, the entire contents of which are incorporated in this application by reference.

### BACKGROUND OF THE DISCLOSURE

Eons ago, stress was pervasive in day-to-day life in a very real, visceral way: either you fought for survival, or you didn't survive. It was a time of incredible simplicity to understand needs and threats. Concerns were mainly on whether a person foraged for enough food to live another day and whether a shelter was safe enough to protect from predators or competitors.

Today, people are more cognizant that stress has many different origins and causes. Instead of directly occurring due to confronting predators or aggressors, stress appears in everyday, seemingly common tasks, such as taking care of family, handling an apparently endless workload, and, to a more pointed effect, trying to make ends meet. Some stress can be beneficial, offering a boost that increases drive and produces energy to push someone through an event like an exam or a work deadline.

As tasks pile up, however, the body begins to treat these concerns as threats, triggering the hypothalamus, a portion of the brain. Nerve and hormonal signals prompt the adrenal glands to release a surge of hormones to deal with any impending threat, releasing adrenaline and cortisol. Adrenaline increases a person's heart rate and elevates blood pressure, among other functions, creating the sensation known as fight-or-flight. Cortisol, known as the primary stress hormone, increases sugar in the bloodstream, enhances the brain's use of these sugars, and has substances on standby to repair tissues. However, cortisol also curbs any functions that may be non-essential or detrimental in a fight-or-flight situation, including immune system responses, mood, motivation, fear, and suppressing the digestive and reproductive systems.

Prolonged or severe stress has been demonstrated to weaken the immune system, damage memory cells, impair concentration, strain the heart, and affect weight gain, specifically with how the body distributes and deposits fat. Stress is implicated in aging, heart disease, depression, diabetes, arthritis, and several illnesses.

Originally, researchers studied the effects physical stress had on the body. Towards the beginning of the millennium, focus shifted to the effects psychological stress had on a person's health. As a result, researchers were able to link psychological stress to increased vulnerabilities to disease and susceptibility to the immune system. Symptoms of chronic stress may include anxiety, depression, headaches, back pain, abdominal pain, lack of sleep, hypertension, and panic attacks, among other physical, cognitive, emotional, and behavioral symptoms.

Usually, the body's stress-response system is self-limiting, with hormone levels returning to normal once a perceived threat passes. As cortisol and adrenaline levels drop, the body's blood pressure and heart rate return to how they were before the threat manifested. However, when these perceived threats are ever-present, the fight-or-flight response continues.

Therefore, it is incredibly important to be able to identify and react to stress in a way to avoid prolonged exposure to stress and mitigate chronic stress symptoms. Certain strategies include eating a healthy diet; regularly exercising; sleeping properly; practicing relaxation techniques such as yoga, deep breathing, or meditation; and taking time to enjoy life or feeling fulfilled, whether in the form of fostering friendships, volunteering, or pursuing a hobby. Combined with medical treatment or physician supervision, these strategies can be helpful to allaying chronic stress.

Currently, most sentiment analysis is based on self-reporting, such as identifying a personality type, how someone feels they are responding to treatment, and adjusting treatment from there. However, this does not help a person who may have a variety of factors for why they feel the way they do. There is a need to move away from subjective reporting of symptoms to a system that facilitates objective, data-driven recommendations that facilitate the treatment process.

### SUMMARY OF THE DISCLOSURE

What is needed, therefore, is a method or process of implementing these practices while helping the medical community, individuals, employers, payers, or health insurance companies follow and trace that progress with a full suite of tools to do so. This includes an open, intelligent behavioral health platform that may pair with wearable biosensors to initiate, encourage, record, and report progress over any period of time. Chronic stress conditions are targeted and clinical protocols developed to effectively mitigate and curb those targeted conditions. Further, what is needed is a system that may allow a user to understand stress triggers and gain voluntary control over automatic body functions, wherein a user may gain the ability to control her body's physiological response to stress. An employer, physician, doctor, or medical professional can then use the information to develop a personalized and targeted treatment plan while being able to access the user's results and use history. They can also compare to other similarly situated users to see what feedback they gave or received and whether any alterations should be made to the current course of treatment.

Users are incentivized both internally and externally to continue to progress with their treatment, being regularly reminded to engage in said treatment, and being guided, whether on their own or by a physician, to treat themselves. Meanwhile, those interested in tracking a user's progress is able to do so on a regular basis, with detailed information being generated for each session the user engages in. The ultimate goal is to help people manage stress related conditions, such as chronic pain or anxiety disorders, by helping users reduce the negative physiological effects of stress.

Accordingly, the present disclosure relates generally to symptom management. More specifically, the disclosure describes a system that pairs biomonitoring mechanisms with symptom management techniques. In some aspects, the symptom management techniques may comprise engaging user activities that may directly or indirectly affect one or more symptoms. In some embodiments, a user may be diagnosed with a disease, infection, or disorder, and the symptom management system may manage and monitor a predefined set of symptoms associated with the diagnosis. In some implementations, individual symptoms may be identified and subsequently monitored and managed separately.

The present disclosure relates to a system of one or more computers that may be configured to perform particular operations or actions by virtue of having software, firmware, hardware, or a combination of them installed on the system that, in operation, causes or cause the system to perform the actions. One or more computer programs can be configured to perform particular operations or actions by virtue of including instructions that, when executed by data processing apparatus, cause the apparatus to perform the actions. One general aspect includes a system for determining a symptom management score, where the system includes a display, one or more wireless communication interfaces, one or more memory resources, which may include a user profile database, a symptom management score database, and one or more processors.

In some aspects, the one or more processors may be configured to receive a first user profile from the user profile database including at least a first symptom of a user and a first subjective rating of the first symptom. In some embodiments, the one or more processors may be configured to receive a first monitor data, where the first monitor data provides a first objective rating of the first symptom. In some implementations, the one or more processors may be configured to receive first programming data, where the first programming data provides a second objective rating of the first symptom. In some embodiments, the one or more processors may be configured to receive a second subjective rating of the first symptom. In some aspects, the one or more processors may be configured to access the symptom management score database. In some implementations, the one or more processors may be configured to transmit the at least first symptom, first subjective rating, first objective rating, second objective rating, and second subjective rating. In some embodiments, the one or more processors may be configured to assign a first symptom management score to the user based on the at least first symptom, first subjective rating, first objective rating, second objective rating, and second subjective rating, where the first symptom management score rates the user ability to manage the first symptom.

Other embodiments of this aspect include corresponding computer systems, apparatus, and computer programs recorded on one or more computer storage devices, each configured to perform the actions of the methods. Implementations may include one or more of the following features. In some embodiments, the one or more processors may be configured to receive a second monitor data, where the second monitor data provides a third objective rating of the first symptom; receive second programming data, where the second programming data provides a fourth objective rating of the first symptom; receive a third subjective rating of the first symptom; access the symptom management score database; transmit the third objective rating, fourth objective rating, and third subjective rating; and assign a second symptom management score to the user based on the at least first symptom, first subjective rating, first objective rating, second objective rating, and second subjective rating, third objective rating, fourth objective rating, and third subjective rating.

In some aspects, the first programming data may further provide the second subjective rating. In some implementations, the one or more processors may be further configured to receive a second symptom of the user, where one or more of the first user profile, first monitor data, and the first programming data provide one or more objective ratings and subjective ratings of the second symptom; and assign a second symptom management score to the user based at least in part on the second symptom and the one or more objective ratings and subjective ratings of the second symptom, where the second symptom management score rates the user ability to manage the second symptom. In some embodiments, the first symptom and the second symptom may comprise symptoms of a first condition, disease, or illness. In some implementations, the one or more processors may be further configured to assign a third symptom management score to the user based at least in part on the first symptom management score and the second symptom management score, where the third symptom management score rates the user ability to manage the first condition, disease, or illness. In some embodiments, the first symptom may comprise a symptom of a first condition, disease, or illness, and the second symptom may comprise a symptom of a second condition, disease, or illness.

In some aspects, the first monitor data may be received from a wearable monitor. In some embodiments, the first monitor data may be received from a medical monitor. In some implementations, the first symptom may comprise pain. In some aspects, the one or more processors may be further configured to receive a direct user input including a third subjective rating of the first symptom. In some embodiments, the one or more processors may be further configured to receive an indirect user input including a third subjective rating of the first symptom. Implementations of the described techniques may include hardware, a method or process, or computer software on a computer-accessible medium.

One general aspect includes a system for developing custom symptom management programming, where the system is configured to: one or more wireless communication interfaces; one or more memory resources including: a user profile database; a symptom management score database; and one or more processors. In some aspects, the one or more processors may be configured to receive a user profile including at least a first symptom and a first subjective rating of the first symptom. The system also includes transmit programming assessing prompts. In some embodiments, the one or more processors may be configured to receive user responses to the programming assessing prompts. The system also includes access a symptom management programming database. In some implementations, the one or more processors may be configured to associate the user responses with symptom management programming criteria, where the symptom management programming criteria includes threshold parameters that determine whether a symptom management program will be effective. In some embodiments, the one or more processors may be configured to identify effective symptom management programming types based at least in part on the user responses. In some aspects, the one or more processors may be configured to associate the user profile with the effective symptom management programming types. Other embodiments of this aspect include corresponding computer systems, apparatus, and computer programs recorded on one or more computer storage devices, each configured to perform the actions of the methods.

Implementations may include one or more of the following features. In some embodiments, the one or more processors may be further configured to receive indirect programming assessing data; and associate indirect programming assessing data with symptom management programming criteria, where the identifying of the effective symptom management programming types is further based at least in part on the indirect programming assessing data. In some aspects, the indirect programming assessing data may include data from social media outlets. In some implementations, the indirect programming assessing data may include data from a wearable monitor. In some embodiments, the indirect programming assessing data may include data from a medical monitor. In some aspects, the one or more processors may be configured to receive direct programming assessing data from an external source. The system where the external source may include a medical professional. The system where the external source may include a monitoring device. Implementations of the described techniques may include hardware, a method or process, or computer software on a computer-accessible medium.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates an exemplary symptom management system.

FIG. 2 illustrates an exemplary symptom management system for providing custom programming.

FIG. 3 illustrates an exemplary symptom management system for identifying a symptom management score.

FIG. 4 illustrates exemplary medical monitoring mechanisms, according to some embodiments of the present disclosure.

FIG. 5 illustrates exemplary wearable monitors, according to some embodiments of the present disclosure.

FIG. 6 illustrates exemplary method steps, according to some embodiments of the present disclosure.

FIG. 7 illustrates exemplary method steps, according to some embodiments of the present disclosure.

FIG. 8 illustrates exemplary graphical user interfaces (GUI) for a stress symptom management activity.

FIG. 9 illustrates exemplary graphical user interfaces (GUI) for symptom management activity for management of benign paroxysmal positional vertigo (BPPV).

FIG. 10 illustrates an exemplary block diagram of an embodiment of a mobile device.

FIG. 11 illustrates an exemplary processing and interface system, according to some embodiments of the present disclosure.

### DETAILED DESCRIPTION

The present disclosure provides generally for symptom management. More specifically, the disclosure describes a system that pairs biomonitoring mechanisms with symptom management techniques.

In the following sections, detailed descriptions of examples and methods of the disclosure will be given. The description of both preferred and alternative examples, though thorough, are exemplary only, and it is understood that, to those skilled in the art, variations, modifications, and alterations may be apparent. It is therefore to be understood that the examples do not limit the broadness of the aspects of the underlying disclosure as defined by the claims.

### Glossary

Medical Monitor: as used herein refers to biosensor and medical devices that may be generally accessible and marketed to health professionals. For example, medical monitors may include electrocardiographs, magnetic resonance imaging machines, or electroencephalograph, as non-limiting examples.
Wearable Monitor: as used herein refers to biosensor devices accessible and marketed to consumers. For example, wearables may include smart watches, fitness monitors, heart monitors, as non-limiting examples.
Symptom: as used herein refers to any departure from normal function or feeling. In some aspects, symptoms may be related to a condition, disease, or illness, such as fibromyalgia, cancer, diabetes, mental health disorders, or bruxism, as non-limiting examples. In some embodiments, symptoms may be related to an incident or activity, such as a car accident, sports participation, or stress.
Symptom Management: as used herein refers to methods and techniques that may alleviate, limit, reduce, or control one or more symptom.
Symptom Management Score: as used herein refers to a standardized score that rates a user's symptoms and their ability to manage those symptoms. In some aspects, the symptom management score may consider a mix of objective and subjective ratings, which may be ascertained directly or indirectly from the user. In some embodiments, a symptom management score may evolve over time, wherein the evolving score may account for management of the symptom and any changes in the quality of the symptom. In some implementations, a symptom management score may consider multiple symptoms, which may be related to the same or different conditions.

The present disclosure relates generally to symptom management. More specifically, the disclosure describes a system that pairs biomonitoring mechanisms with symptom management techniques. In some aspects, the symptom management techniques may comprise engaging user activities that may directly or indirectly affect one or more symptoms. In some embodiments, a user may be diagnosed with a disease, infection, or disorder, and the symptom management system may manage and monitor a predefined set of symptoms associated with the diagnosis. In some implementations, individual symptoms may be identified and subsequently monitored and managed separately.

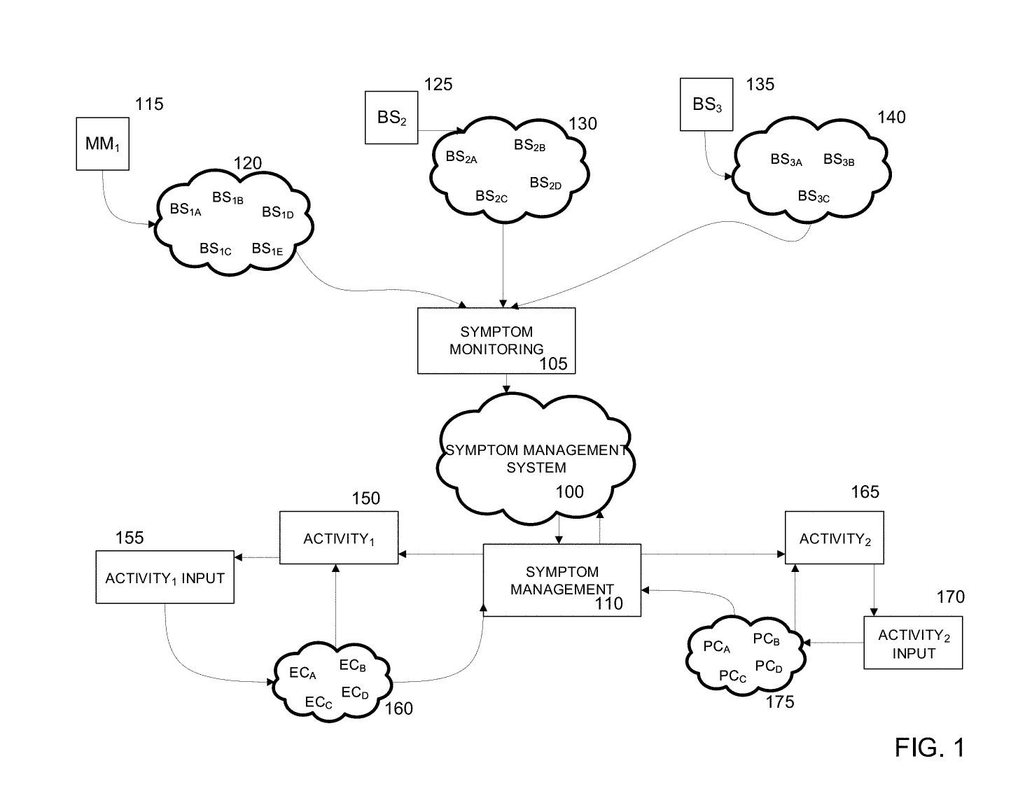

Referring now to FIG. 1, an exemplary symptom management system 100 is illustrated. In some aspects, the symptom management system 100 may comprise a symptom monitoring system 105 and a symptom management system 110, wherein data may be exchanged periodically to allow for dynamic customization of the symptom management system 100. In some embodiments, the period for exchange of data may be frequent, and the customization may occur in real time, wherein the symptom management activity may adjust as symptom monitoring data may be received. In some implementations, the period for exchange of data may occur on a predefined frequency, which may be set by the user or healthcare professional.

In some aspects, the symptom monitoring system 105 may receive monitor data 120, 130, 140 from one or more monitors 115, 125, 135. In some aspects, the monitors 115, 125, 135 may comprise a mix of medical monitors 115, 135 and wearable monitors 125. In some embodiments, the symptom management system 100 may be set up and controlled in part or entirely by a healthcare professional, such as a doctor, healthcare center, therapist, nurse, as non-limiting examples. In some aspects, symptom activities may be self-guided, such as based on user input, assessments, self-evaluations, and biosensor data.

As an illustrative example, a user may have cancer, wherein the user may be monitored by multiple healthcare providers, such as oncologists, psychiatrists, therapists, and pain management physicians.

In some aspects, the monitors 115, 125, 135 may independently collect and transfer monitor data 120, 130, 140 to the symptom monitoring system 105. In some aspects, a tertiary device may interface with the monitors 115, 125, 135, wherein the tertiary device may perform one or more function, such as access the monitor data 120, 130, 140; control monitor 115, 125, 135 settings; verify, interpret, or monitor data 120, 130, 140, as non-limiting examples. In some embodiments, the tertiary device may interface with the monitors 115, 125, 135, wherein the tertiary device may perform one or more function, such as access the monitor data 120, 130, 140; control monitor 115, 125, 135 settings; verify, interpret, or monitor data 120, 130, 140, as non-limiting examples.

As an illustrative example, a user may have had knee replacement surgery, wherein the implant by an orthopedic surgeon may comprise a sensor. The comparative use between legs may be monitored for overcompensation by a physical therapist, back pain monitors, and fitness monitors. If a user is known to clench teeth when in pain (which may cause headaches), facial muscle monitors may be utilized to monitor bruxism.

In some embodiments, the tertiary device may be an intermediary device that has complete or limited control over transfer of monitor data 120, 130, 140. In some implementations, the tertiary device may passively interface with one or more monitors 115, 125, 135 and monitor data 120, 130, 140. For example, the tertiary device may be an internal system at a healthcare facility, which may accept medical monitor data 120, 140.

In some embodiments, a symptom management system 110 may transmit activity instructions 150, 165, wherein participation in the activity may directly or indirectly reduce one or more symptom. For example, an activity may comprise a breathing exercise, and the symptom may be anxiety, wherein participating in the breathing exercise may reduce anxiety. As another example, the symptom may be back pain, and the activity may comprise neck exercises, wherein participation in the neck exercises may increase user awareness of positions or actions that may exacerbate the back pain.

In some aspects, the activities may comprise a mix of functions and interactions. For example, a first activity 150 may prompt a user to perform specific exercises, wherein a monitor may verify or track activity input 155. For example, a monitor may comprise neck muscle sensors, and the activity may prompt a user to move his head in particular directions, wherein the sensors may verify that the user is performing the activity correctly. In some embodiments, a second activity 165 may comprise a game, wherein the activity input 170 may comprise user interaction with a smartphone interface.

In some aspects, activity data 160, 175 may be collected from one or more activities 150, 165 and activity input 155, 170, wherein activity data 160, 175 may be transmitted to the symptom management system 110. A tertiary device may interact with one or more activities 150, 165, activity input 155, 170, or activity data 160, 175. In some aspects, a tertiary device may tailor activity parameters, such as based on known limitations of the user, applied limitations from a healthcare provider, or other third party criteria. In some aspects, activity parameters for programs, games, and content may be dynamically customized, such as through self-rating, self-assessments, and symptom data changes.

For example, symptoms of a user may prompt a movement-based activity, such as rehabilitation after a knee replacement. The specific movement limitations may be set by a physical therapist, and adjusted as the user gains strength in her knee. The physical therapist may review activity input data and assess user progress and adjust the activity parameters accordingly. In some aspects, a user may suffer generally from stress-related symptoms, wherein the symptom management system may depend primarily on self-management.

In some aspects, symptom management system data may be aggregated over population groups, wherein the data may be used to understand symptom management trends, for predictive analysis, or other uses. For example, stress levels may be monitored over populations over time, wherein symptoms may be evaluated based on aggregated symptom data and usage and engagement data. In some aspects, metadata may be aggregated, such as geospatial and time data, wherein the metadata may be collected anonymously. In some implementations, a user may compare their individual symptom data, symptom management data, and engagement data to population groups, such as others with similar diagnosis.

In some aspects, symptom management system data may be aggregated as a method of understanding effectiveness of a particular drug or treatment. Pharmaceutical companies or medical device companies may use the data as support or indications of the expected effectiveness for predefined populations, such as within an age group with similar diagnoses. In some aspects, effectiveness of a medical device or treatment may be tracked by an individual basis, wherein a user and/or health care provider can monitor the effectiveness of a particular product or treatment, such as light or electromagnetic treatments.

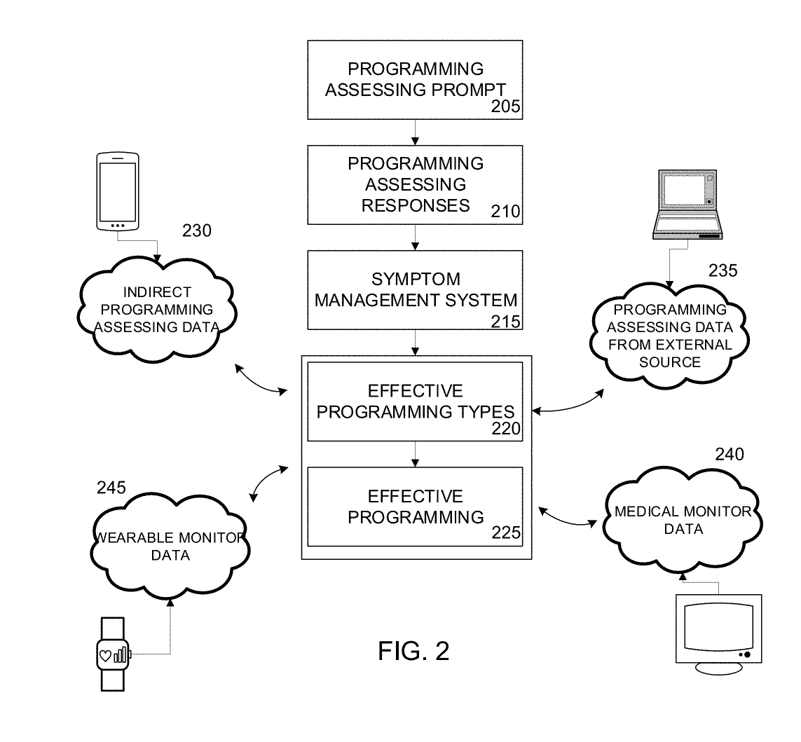

Referring now to FIG. 2, an exemplary symptom management system 215 for developing and providing custom programming 220, 225 is illustrated. In some aspects, programming assessment prompts 205 may be transmitted to a user, and the user programming assessment responses 210 may be transmitted to the symptom management system 215. In some embodiments, the symptom management system 215 may comprise a database with programming, wherein the programming may be associated with programming criteria. In some implementations, the programming criteria may comprise threshold parameters that may determine whether particular programming or programming types may be effective with a user. For example, if a user normally does not sit still, walking meditations may be incorporated into their programming. By way of another example, a user with anxiety may receive programming for quelling constant racing thoughts. In some embodiments, programming may be organized into programming types.

In some implementations, programming types may be organized based on a range of characteristics, such as symptoms the programming could help manage, activity requirements, and audio levels, as non-limiting examples. For example, a user profile may comprise at least one symptom, and the symptom management system 215 may filter the programming by symptom, wherein programming unrelated to the symptom may be eliminated from selection. In some embodiments, the programming assessment prompts 205 may relate to preferences, such as when symptom management may be convenient for the user or preferred aesthetics.

In some implementations, the programming assessment prompts 205 may be related to the symptom, such as the severity, any diagnosed conditions that may contribute to the symptom, related symptoms, exacerbating circumstances, and alleviating circumstances, as nonlimiting examples. For example, a user with post-traumatic stress disorder may receive exercises that avoid loud noises or other triggers. In some aspects, the programming assessment prompts 205 may indirectly relate to the programming. For example, the programming assessment prompts 205 may ask about personality type or prompt open input of sentences, wherein a natural language analysis may be applied.

In some aspects, the symptom management system 215 may further receive other types of data that may be considered when determining effective programming 225 and effective programming types 220. In some embodiments, the symptom management system 215 may receive indirect data 230, external source data 235, medical monitor data 240, and wearable monitor data 245.

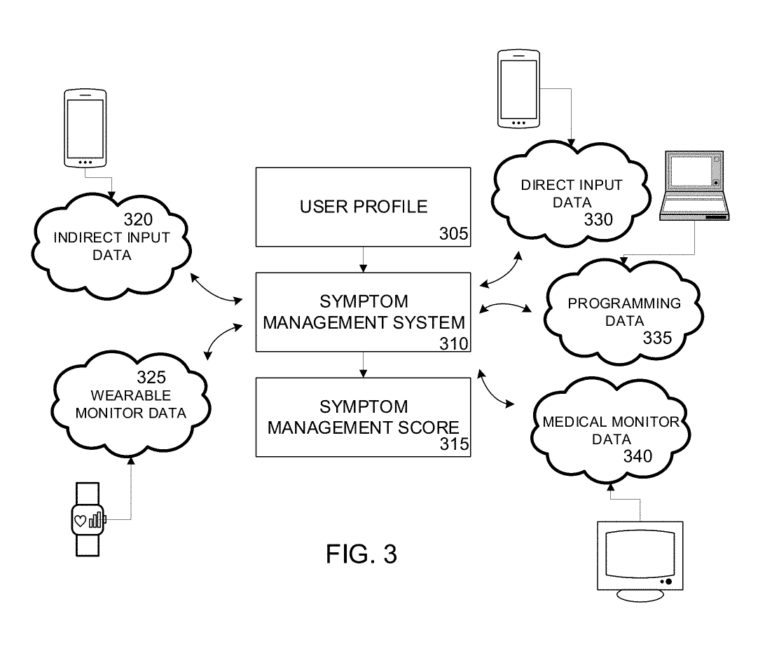

Referring now to FIG. 3, an exemplary symptom management system 310 for assessing a symptom management score 315 is illustrated. In some aspects, the symptom management system 310 may receive a user profile 305, which may comprise base details and at least one symptom that the user may want to manage. In some embodiments, the user profile 305 may include one or more subjective ratings of the symptom, such as through direct self-reporting. In some aspects, a score may be based on progress, which requires an initial and at least a second evaluation to determine how a user is managing a symptom. For example, if a user originally reports depression as “very sad” and then through programming it is determined that the user is “not unhappy,” then the symptom management score 315 would show there has been some progress in measuring that symptom of sadness. In some embodiments, it may be important to get an initial objective and subjective ratings as well as follow ups on these ratings. This way, even if a patient is objectively improving, the score still accounts for the user's perception of progress and the symptom.

In some implementations, the symptom management system 310 may receive one or more subjective ratings and the objective ratings from indirect input data 320, wearable monitor data 325, direct input data 330, programming data 335, and medical monitor data 340. In some embodiments, the data may be acquired over time, wherein the symptom management score 315 may evolve over time, which may indicate how a user may be getting better or worse at managing symptoms. In some aspects, a separate symptom management score 315 may be calculated for different symptoms. In some implementations, multiple symptoms may be grouped by condition or illness, and a symptom management score 315 may assess the user's ability to manage all the symptoms associated with the condition or illness. In some aspects, such as where a user may have a primary illness and comorbidities, multiple symptom management scores 315 may be provided and viewed, such as one that relates to an overall assessment, ones that relates to symptoms associated with the primary illness and each comorbidity, and ones that relate to main symptoms, as non-limiting examples.

For example, the symptom management system may be allowed to access social media accounts to survey the natural language used in a user's day to day life. By compiling this information across various accounts, the symptom management system may compare to model use cases or research cases to assign a score for a propensity for certain mental health conditions. As a result of this, the symptom management system can then begin to develop customized programming for the user, as previously detailed above.

In some implementations, the symptom management system 310 may receive geolocation data or location data input by a user, like a zip code or address by way of example, to determine whether there is a stress level associated with that zip code or address. In some aspects, this information may be pulled from publicly available information, such as census data as a non-limiting example. In some embodiments, geolocation data may be considered to create a more comprehensive symptom management score 315, particularly early on, when the symptom management system 310 may have limited subjective and objective ratings from a particular user.

As an illustrative example, a user may be a female of an age and occupation associated with high stress, live in an area with a highly stressed population and make a two-hour commute to, and from work every day through heavy traffic. Typically, an extensive commute through traffic increases stress on a person. With this geolocation and demographic data coupled with initial subjective and objective ratings, a user may be rated with a symptom management score 315 that indicates high stress levels. Over time, such as through receipt of monitoring data 325, 340 and programming data 335, the symptom management system 310 may deduce that the extensive commute is actually a relaxing period for the user.

For example, the user may enjoy the commute because it is time alone where she listens to her favorite music. Similarly, the symptom management system 310 may deduce that only the fifteen minutes prior to reaching the destination, either home or work, are highly stressful to the user, as she anticipates the stress of work and home life. The symptom management system 310 may also deduce that stress levels are heavily increased if the commute exceeds two hours, meaning the user may be late for work or after work commitments. Accordingly, in some aspects, geographic, commute, and demographic data may be used as foundation for the symptom management score 315 and then tailored over time as further objective and subjective ratings are gathered.

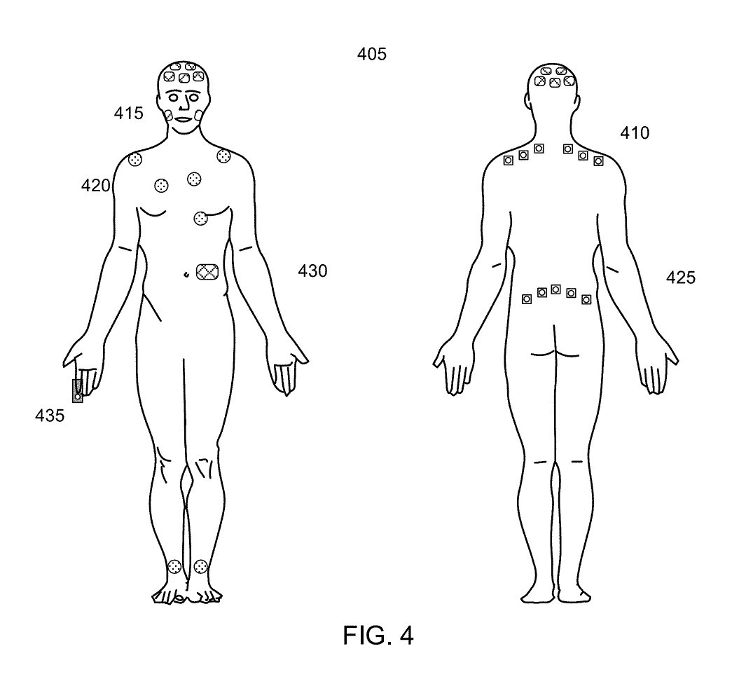

Referring now to FIG. 4, exemplary medical monitoring mechanisms are illustrated, wherein the medical monitoring mechanisms may be integrated into a symptom management system. In some aspects, electrodes 405 may be placed on the head of a user in preparation for an electroencephalogram, which may monitor brain activity. In some embodiments, electrocardiogram contacts 420 may be placed to monitor heart activity.

In some implementations, a pulse oximeter 435 may be placed on a user finger, wherein a plethysmograph may be located at a health care facility or a personal device may be wearable. In some aspects, a user may be diabetic and wear an insulin pump 430, which may collect data periodically, such as dosage information, glucose levels, or power supply data, as non-limiting examples. In some embodiments, facial muscle sensors 415, neck muscle sensors 410, and lower back muscle sensors 425 may be placed on a user, which may be used to monitor muscle activity. For example, muscle sensors 410, 415, 425 may be utilized to monitor stress levels, as many individuals engage their neck and jaw muscles in response to stress, and activities that may force or teach a user to relax their muscles may allow for a reduction in stress.

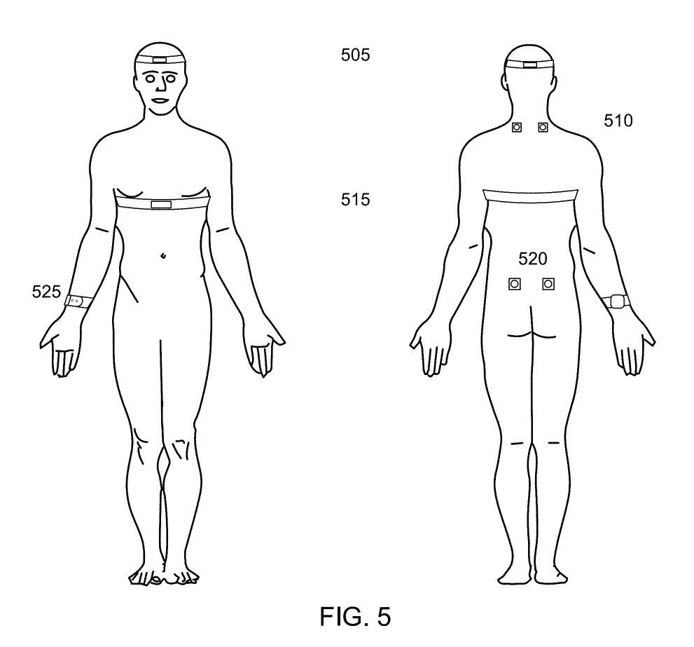

Referring now to FIG. 5, exemplary wearable monitors 505, 510, 515, 520, 525 are illustrated, wherein the wearable monitors 505, 510, 515, 520, 525 may be integrated into a symptom management symptom. In some aspects, a user may wear a head monitor 505, such as during sleep, rest, or during symptom management activity engagement. In some embodiments, a user may wear a heart monitor 515, such as during exertive exercises, sleep, or symptom management activity engagement. In some aspects, a user may wear a fitness monitor 525, such as a smartwatch, Fitbit, Misfit, Jawbone, Moov, or Leaf, as non-limiting examples. In some implementations, a user may utilize wearable muscle sensors 515, 520.

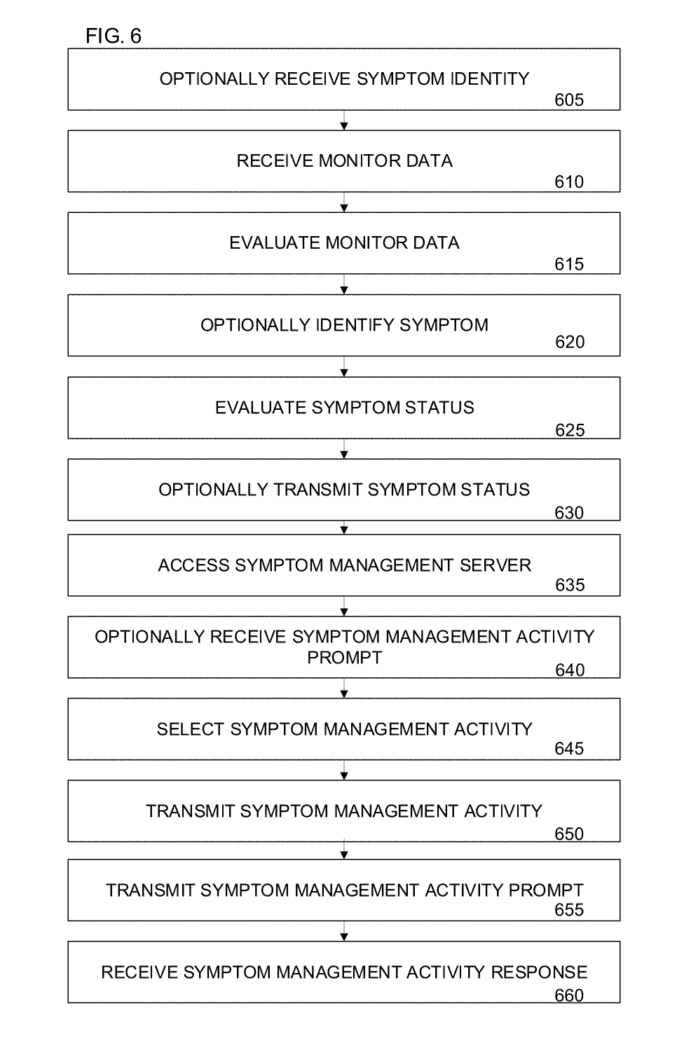

Referring now to FIG. 6, exemplary method steps are illustrated. In some embodiments, at 605, symptom identification may be received. At 610, monitor data may be received, and at 615, received monitor data may be evaluated. In some aspects, at 620, a symptom may be identified, such as from evaluating monitor data at 615. At 625, symptom status may be evaluated. In some embodiments, at 630, symptom status may be transmitted. At 635, a symptom management server may be accessed.

In some embodiments, at 640, symptom management activity instruction may be received, such as from a healthcare provider or user. At 645, a symptom management activity may be selected. At 650, a symptom management activity may be transmitted, such as to a user or preliminarily to a third party, such as a healthcare provider or a payment vendor, as non-limiting examples. For example, a user may be required to submit a payment for an activity, or a healthcare provider may need to approve an activity. In some aspects, at 655, a symptom management activity prompt may be transmitted. At 660, symptom management activity data may be received. In some aspects, symptom and symptom status evaluation at 615, 625 may be periodic and simultaneous with symptom management activity transmission at 650 and receipt of symptom management activity at 660.

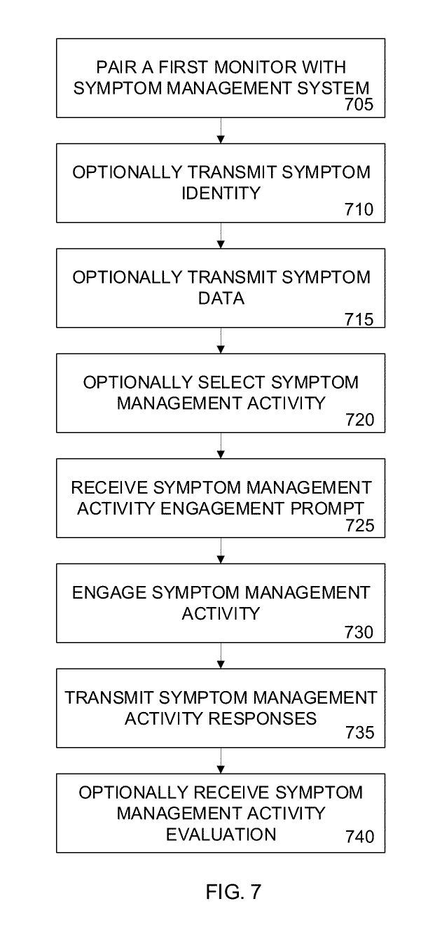

Referring now to FIG. 7, exemplary method steps are illustrated. In some aspects, at 705, a first monitor may be paired with a symptom management system. In some embodiments, at 710, symptom identification may be transmitted. In some embodiments, at 715, symptom data may be transmitted, such as manual input of user complaints or automatic input of symptoms generally associated with a diagnosis. In some aspects, at 720, symptom management activity may be selected, such as by a healthcare provider or user.

At 725, symptom management activity prompt may be received. At 730, symptom management activity may be engaged. At 735, symptom management activity responses may be transmitted. In some implementations, at 740, symptom management activity evaluation may be received. For example, a user may receive an evaluation of his performance during a symptom management activity, wherein the evaluation may be based on a comparison of symptom status before, during, and after the engagement.

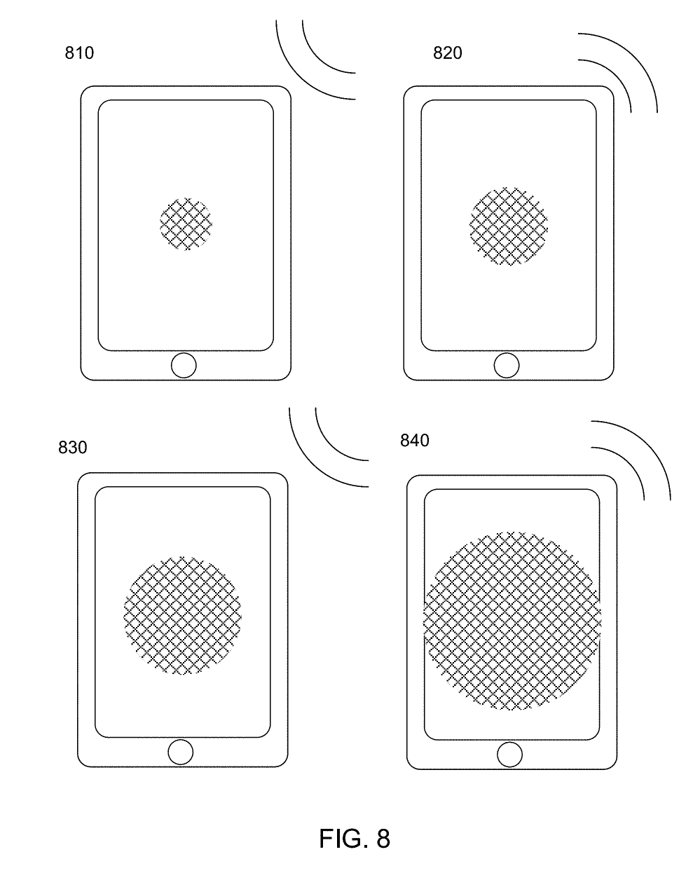

Referring now to FIG. 8, exemplary graphical user interfaces (GUI) 810, 820, 830, 840 for a stress symptom management activity is illustrated. In some aspects, the symptom management activity may be related to a breathing exercise, wherein a user may breathe in concert with a visual cue in the GUI 810, 820, 830, 840. In some embodiments, the portable device may receive and transmit symptom management data. In some aspects, the symptom management system may adjust the symptom management activity in response to changing symptoms, such as received from the monitors. For example, a user may be wearing facial and neck muscle monitors and heart monitor, and the breathing activity may be prolonged or shortened and the speed of the breathing may quicken or slow based on data from the worn monitors.

In some aspects, a user may engage with symptom management activities prior to, during, or after typical stress-inducing situations, such as work, sports, public speaking engagements, or performances. In some aspects, an employer may provide perks or require their employees to engage with symptom management activities, wherein an employer may monitor the general stress levels of its employees. In some implementations, an employer may evaluate the effectiveness of particular company policies and equipment, such as safety settings or computer desks.

In some aspects, a GUI 810, 820, 830, 840 may be paired with audio, which may be customizable, adaptive, static, or combinations thereof. In some embodiments, audio may dynamically change based on user response, such as through direct user input, changes in symptom data received from monitors, or symptom management activity responses. Audio may comprise music, sounds, tones, rhythmic beats, or combinations thereof.

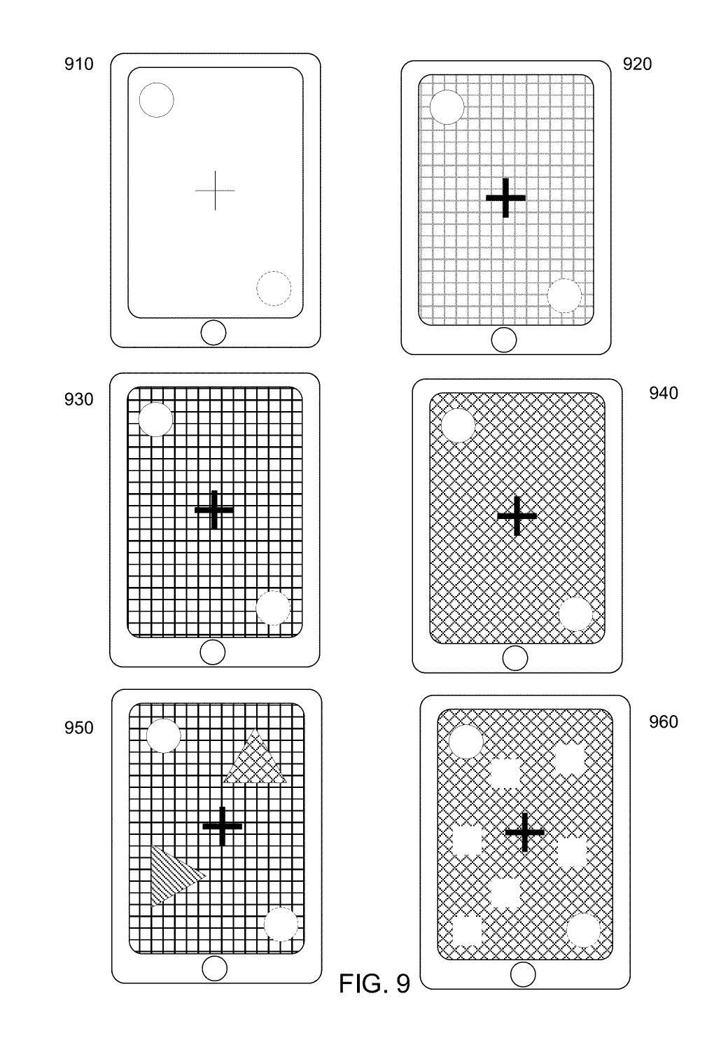

Referring now to FIG. 9, exemplary graphical user interfaces (GUI) 910-960 for symptom management activity for management of benign paroxysmal positional vertigo (BPPV). In some aspects, the initial GUI 910 may present two circles that a user must switch between to engage with the symptom management activity. In some embodiments, the portable device may recognize eye placement, such as through wireless communication with a sensor in an ophthalmic lens device. In some embodiments, a user may physically engage with the GUI 910, wherein the exercise progresses as the user taps different portions of the GUI 910.

As a dynamic treatment for BBPV, the GUI 910-960 may present exercises of increasing difficulty, such as through adding a busier background or other shapes. In some implementations, a user may be monitored through periodic electronystagmograms (ENG) or videonystagmograms (VNG), wherein the data may be integrated into the symptom management system. For example, the difficulty levels of the symptom management activity may be adjusted automatically based on receipt of the ENG or VNG results. As another example, the difficulty levels may be manually configured by a healthcare professional based on their evaluation of the ENG or VNG results.

As an illustrative example, a user may have a cardiac infarction, stent implanted as a mechanism to limit their hypertension. In some aspects, the stent may be configured to collect and transmit data, such as predefined ambient characteristics, including for example temperature, width of the blood vessel, elasticity of the vessel walls, or blood flow speed, or predefined characteristics of the stent itself, including for example, date of insertion or durability characteristics.

The data may be collected remotely from an external device, such as a portable device or a data access center at a cardiology facility. A user may go to a health care provider periodically for an ECG, wherein ECG data may be transmitted to the symptom management system. In some aspects, a healthcare provider may initiate a symptom management activity for the user to engage with before, during, and after the ECG.

The user may utilize wearable monitors, which may be worn throughout the day or during active hours. For example, a user may wear a heart monitor during exercise, and a general fitness monitor at all times, wherein the general fitness monitor may estimate activity and heart rate. Collecting data from a range of wearable monitors and medical monitors may provide a more accurate understanding of the status of the symptoms.

As an illustrative example, a user may be epileptic. The user may be monitored with an encephalelectrocardiogram (EEG) every three months in a hospital. A health facility may administer symptom management exercises to the user before, during, after, or a combination thereof, the EECG and EEG, wherein the symptom management exercises may be customized to the user based on prescribed parameters. In some aspects, prescribed parameters may be adapted for personal symptom management activities, which may be engaged on a smartphone or other personal portable device.

The user may be monitored with an electrocardiogram every month, and the user may be prescribed a personal medical monitoring device, such as an implant or one that may be carried. A health professional may recommend the user also utilize a consumer heart monitor during exertive activities, and a fitness monitor at all times, as a secondary mechanism to monitor.

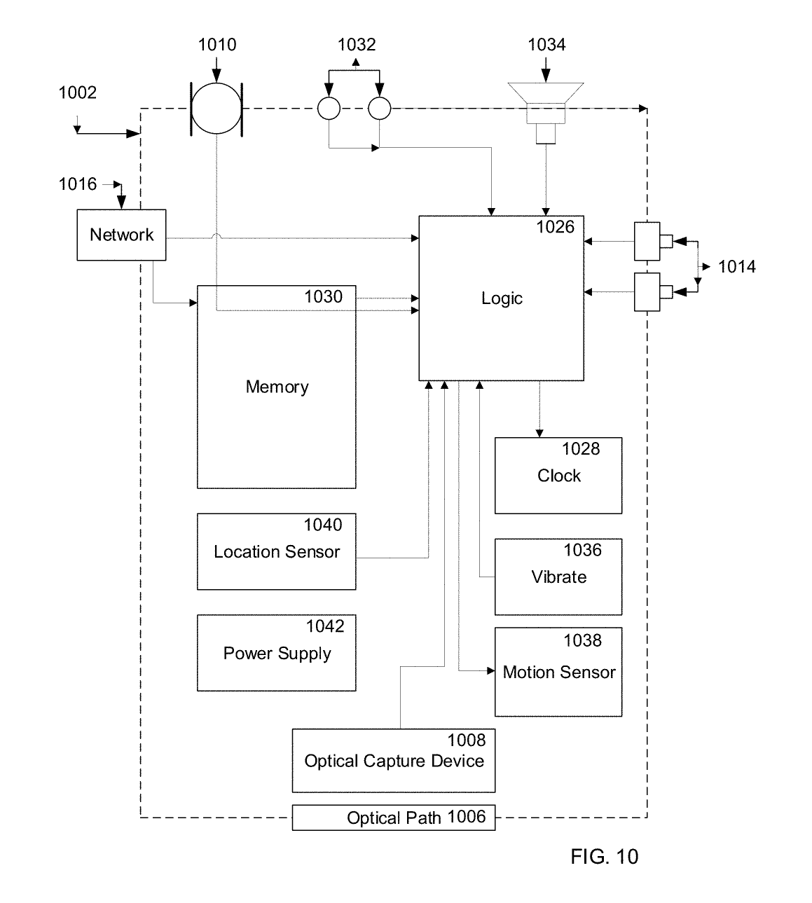

Referring now to FIG. 10, an exemplary block diagram of an exemplary embodiment of a mobile device 1002 is illustrated. The mobile device 1002 may comprise an optical capture device 1008, which may capture an image and convert it to machine-compatible data, and an optical path 1006, typically a lens, an aperture, or an image conduit to convey the image from the rendered document to the optical capture device 1008. The optical capture device 1008 may incorporate a Charge-Coupled Device (CCD), a Complementary Metal Oxide Semiconductor (CMOS) imaging device, or an optical sensor of another type.

In some embodiments, the mobile device 1002 may comprise a microphone 1010, wherein the microphone 1010 and associated circuitry may convert the sound of the environment, including spoken words, into machine-compatible signals. Input facilities 1014 may exist in the form of buttons, scroll-wheels, or other tactile sensors such as touch-pads. In some embodiments, input facilities 1014 may include a touchscreen display. Visual feedback 1032 to the user may occur through a visual display, touchscreen display, or indicator lights. Audible feedback 1034 may be transmitted through a loudspeaker or other audio transducer. Tactile feedback may be provided through a vibration module 1036.

In some aspects, the mobile device 1002 may comprise a motion sensor 1038, wherein the motion sensor 1038 and associated circuitry may convert the motion of the mobile device 1002 into machine-compatible signals. For example, the motion sensor 1038 may comprise an accelerometer, which may be used to sense measurable physical acceleration, orientation, vibration, and other movements. In some embodiments, the motion sensor 1038 may comprise a gyroscope or other device to sense different motions.

In some implementations, the mobile device 1002 may comprise a location sensor 1040, wherein the location sensor 1040 and associated circuitry may be used to determine the location of the device. The location sensor 1040 may detect Global Position System (GPS) radio signals from satellites or may also use assisted GPS where the mobile device may use a cellular network to decrease the time necessary to determine location. In some embodiments, the location sensor 1040 may use radio waves to determine the distance from known radio sources such as cellular towers to determine the location of the mobile device 1002. In some embodiments these radio signals may be used in addition to and/or in conjunction with GPS.

In some aspects, the mobile device 1002 may comprise a logic module 1026, which may place the components of the mobile device 1002 into electrical and logical communication. The electrical and logical communication may allow the components to interact. Accordingly, in some embodiments, the received signals from the components may be processed into different formats and/or interpretations to allow for the logical communication. The logic module 1026 may be operable to read and write data and program instructions stored in associated storage 1030, such as RAM, ROM, flash, or other suitable memory. In some aspects, the logic module 1026 may read a time signal from the clock unit 1028. In some embodiments, the mobile device 1002 may comprise an on-board power supply 1042. In some embodiments, the mobile device 1002 may be powered from a tethered connection to another device, such as a Universal Serial Bus (USB) connection.

In some implementations, the mobile device 1002 may comprise a network interface 1016, which may allow the mobile device 1002 to communicate and/or receive data to a network and/or an associated computing device. The network interface 1016 may provide two-way data communication. For example, the network interface 1016 may operate according to an internet protocol or near-field communication, which may communicate with a monitor. As another example, the network interface 1016 may comprise a local area network (LAN) card, which may allow a data communication connection to a compatible LAN. As another example, the network interface 1016 may comprise a cellular antenna and associated circuitry, which may allow the mobile device to communicate over standard wireless data communication networks. In some implementations, the network interface 1016 may comprise a Universal Serial Bus (USB) to supply power or transmit data. In some embodiments, other wireless links known to those skilled in the art may also be implemented.

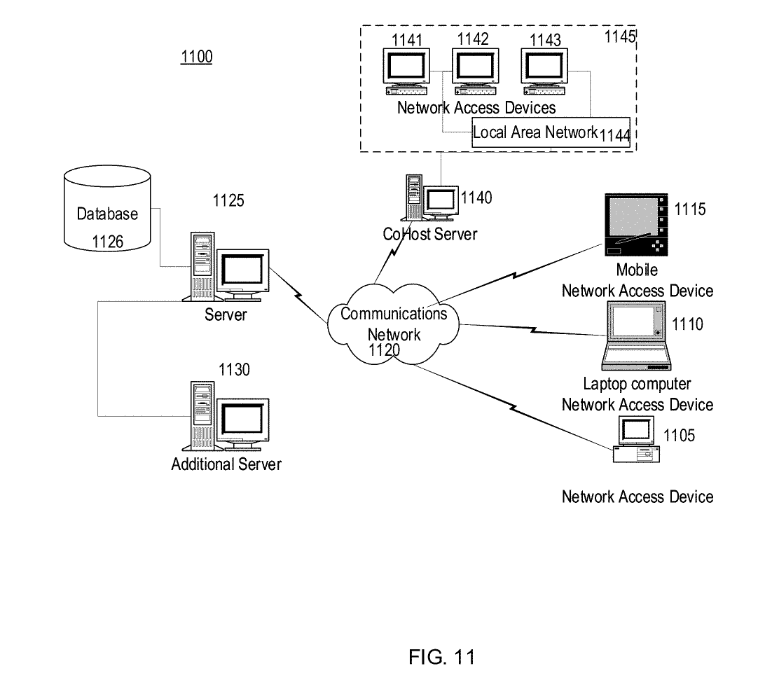

Referring now to FIG. 11, an exemplary processing and interface system 1100 is illustrated. In some aspects, access devices 1115, 1110, 1105, such as a paired portable device 1115 or laptop computer 1110 may be able to communicate with an external server 1125 though a communications network 1120. The external server 1125 may be in logical communication with a database 1126, which may comprise data related to identification information and associated profile information. In some embodiments, the server 1125 may be in logical communication with an additional server 1130, which may comprise supplemental processing capabilities.

In some aspects, the server 1125 and access devices 1105, 1110, 1115 may be able to communicate with a cohost server 1140 through a communications network 1120. The cohost server 1140 may be in logical communication with an internal network 1145 comprising network access devices 1141, 1142, 1143 and a local area network 1144. For example, the cohost server 1140 may comprise a payment service, such as PayPal or a social network, such as Facebook or a dating website.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in a combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. Nevertheless, it will be understood that various modifications may be made without departing from the spirit and scope of the claimed disclosure.

What is claimed is:

1. A system for determining a symptom management score, wherein the system comprises:
a display;
one or more wireless communication interfaces;
one or more memory resources comprising:
a user profile database;
a symptom management score database; and
one or more processors to:
receive a first user profile from the user profile database comprising at least a first symptom of a user and a first subjective rating of the first symptom;
receive a first monitor data, wherein the first monitor data provides a first objective rating of the first symptom;
receive first programming data, wherein the first programming data provides a second objective rating of the first symptom;
receive a second subjective rating of the first symptom;
access the symptom management score database;
transmit the at least first symptom, first subjective rating, first objective rating, second objective rating, and second subjective rating; and
assign a first symptom management score to the user based on the at least first symptom, first subjective rating, first objective rating, second objective rating, and second subjective rating, wherein the first symptom management score rates a user ability to manage the first symptom.

2. The system of claim 1, wherein the one or more processors are further configured to:
receive a second monitor data, wherein the second monitor data provides a third objective rating of the first symptom;
receive second programming data, wherein the second programming data provides a fourth objective rating of the first symptom;
receive a third subjective rating of the first symptom;
access the symptom management score database;
transmit the third objective rating, fourth objective rating, and third subjective rating; and
assign a second symptom management score to the user based on the at least first symptom, first subjective rating, first objective rating, second objective rating, and second subjective rating, third objective rating, fourth objective rating, and third subjective rating.

3. The system of claim 1, wherein the first programming data further provides the second subjective rating.

4. The system of claim 1, wherein the one or more processors are further configured to:
receive a second symptom of the user, wherein one or more of the first user profile, first monitor data, and the first programming data provide one or more objective ratings and subjective ratings of the second symptom; and
assign a second symptom management score to the user based at least in part on the second symptom and the one or more objective ratings and subjective ratings of the second symptom, wherein the second symptom management score rates the user ability to manage the second symptom.

5. The system of claim 4, wherein the first symptom and the second symptom comprise symptoms of a first condition, disease, or illness.

6. The system of claim 5, wherein the one or more processors are further configured to assign a third symptom management score to the user based at least in part on the first symptom management score and the second symptom management score, wherein the third symptom management score rates the user ability to manage the first condition, disease, or illness.

7. The system of claim 4, wherein the first symptom comprises a symptom of a first condition, disease, or illness, and the second symptom comprises a symptom of a second condition, disease, or illness.

8. The system of claim 1, wherein the first monitor data is received from a wearable monitor.

9. The system of claim 1, wherein the first monitor data is received from a medical monitor.

10. The system of claim 1, wherein the first symptom comprises pain.

11. The system of claim 1, wherein the one or more processors are further configured to receive a direct user input comprising a third subjective rating of the first symptom.

12. The system of claim 1, wherein the one or more processors are further configured to receive an indirect user input comprising a third subjective rating of the first symptom.

13. A system for developing custom symptom management programming, wherein the system is configured to:
one or more wireless communication interfaces;
one or more memory resources comprising:
a user profile database;
a symptom management score database; and
one or more processors to:
receive a user profile comprising at least a first symptom and a first subjective rating of the first symptom;
transmit programming assessing prompts;
receive user responses to the programming assessing prompts;
access a symptom management programming database;
associate the user responses with symptom management programming criteria, wherein the symptom management programming criteria comprises threshold parameters that determine whether a symptom management program will be effective;
identify effective symptom management programming types based at least in part on the user responses; and
associate the user profile with the effective symptom management programming types.

14. The system of claim 13, wherein the one or more processors are further configured to:
receive indirect programming assessing data; and
associate indirect programming assessing data with symptom management programming criteria, wherein the identifying of the effective symptom management programming types is further based at least in part on the indirect programming assessing data.

15. The system of claim 14, wherein the indirect programming assessing data comprise data from social media outlets.

16. The system of claim 14, wherein the indirect programming assessing data comprise data from a wearable monitor.

17. The system of claim 14, wherein the indirect programming assessing data comprise data from a medical monitor.

18. The system of claim 13, wherein the one or more processors are further configured to receive direct programming assessing data from an external source.

19. The system of claim 18, wherein the external source comprises a medical professional.

20. The system of claim 18, wherein the external source comprises a monitoring device.
